(19) United States
(12) Patent Application Publication
(10) Pub. No.: US 2011/0307006 A1
(43) Pub. Date: Dec. 15, 2011

(54) VASCULAR ACCESS CLOSURE DEVICES AND METHODS

(76) Inventor: Timothy P. Murphy, Providence, RI (US)

(21) Appl. No.: 12/797,190

(22) Filed: Jun. 9, 2010

(51) Int. Cl. A61B 17/03

(52) U.S. Cl. 606/213

(57) ABSTRACT

Devices and related methods are disclosed that generally involve closing of an arteriotomy using a vascular access closure device that is deployed within the artery and closes the arteriotomy or arterial wound from the inside. The device can be biodegradable and/or self-expandable, and can optionally be formed of a shape memory polymer or a shape memory skeleton having a blood impermeable skin disposed therearound.

### FIELD OF THE INVENTION

The present invention relates to medical devices and methods and more particularly, to vascular access closure. The invention has application, by way of example, in the repair of arteriotomies, e.g., resulting from medical procedures, disease, trauma or other causes.

### BACKGROUND OF THE INVENTION

There is frequent need for vascular access closure in the repair of arteriotomies resulting from medical procedures, disease, trauma or other causes. For example, there are more than twelve million arterial catheter procedures performed annually that require control of bleeding at the access site after the procedure. These procedures are usually performed by obtaining needle access to an artery, usually a femoral, brachial or radial artery. Once the needle is in place and blood returns, a guidewire is placed through the needle into the artery. The needle is then removed and exchanged for a catheter or sheath. A diagnostic or therapeutic procedure is performed, for example, angiography, angioplasty, stent placement, etc. Once the procedure is completed, all catheters and guidewires are removed.

Whether following a catheter procedure or otherwise, a method to arrest hemorrhage, either external or into the soft tissues surrounding the artery, is necessary since arteries are under greater than atmospheric pressure. Traditionally, this has been done by manual compression. For example, pressure can be applied with the fingers over the access site for a variable period, usually 15 minutes. Clotting factors and platelets in the blood form a hemostatic plug during this time in most people.

However, there are occasions when manual compression can fail, resulting in blood loss that can be considerable in the soft tissues or externally. This complication is more frequent for those who are obese, for larger vascular access devices, for those with coagulopathy or who are receiving anticoagulation or antiplatelet therapy, or for those in whom blood pressure is high or must be maintained at elevated levels (for example individuals with subarachnoid hemorrhage and cerebral artery vasospasm).

Alternatives or supplements to manual compression include use of external appliances, such as a five pound sand bag placed over the access site after manual compression. The art also suggests the use of external clamps (e.g., FemoStop®, RADI Medical Devices, Wilmington, Mass.) as the primary method of compression. More recently, a number of devices placed internally within or around the artery have been used to aid or achieve hemostasis after arterial catheter access. Those that are external to the artery include VasoSeal® (Maquet Cardiovascular, Fairfield, N.J.), Mynx 5 (Access Closure, Mountain View, Calif.) and AngioSeal® (St. Jude Medical, St. Paul, Minn.), which are collagen-based materials that are injected along the track of the needle outside of the artery. StarClose® (Abbott Vascular, Abbott Park, Ill.) is a device that applies a clip to the tunica adventitia or outer wall of the artery to “pinch” the arterial wound closed and aid hemostasis, and the PerClose®/ProGlide® (Abbott Vascular) device applies two sutures to opposite sides of the arterial wound (arteriotomy) that are then closed after device removal to suture the wound shut.

Notwithstanding the success of prior art techniques, there is a continual need for improved devices and methods for safely and efficiently achieving vascular access closure.

In view of the foregoing, an object of the invention is to provide improved medical devices and methods.

A related aspect of the invention is to provide such improved medical devices and methods as can be used for vascular access closure.

A further related object of the invention is to provide such improved medical devices and methods as can be applied in the repair of arteriotomies, e.g., resulting from medical procedures, disease, trauma or other causes.

A still further related object of the invention is to provide such improved medical devices and methods as are safe and effective.

### SUMMARY OF THE INVENTION

The foregoing are among the objects attained by the invention, which provides in some aspects devices and methods for closing of an arteriotomy using a vascular access closure device that is deployed within the artery and that closes the arterial wall opening from the inside.

Thus, in one aspect, the invention provides a vascular closure device that includes a body having a first conformation adaptable to pass through an opening in a wall of the vessel and that has a second conformation adaptable to conform to that wall in a vicinity of the opening. The body transitions from the first conformation to the second conformation in response to one or more triggers which can include, by way of example, exposure to body temperature, exposure to moisture, and/or release of a tether.

Related aspects of the invention provide a closure device as described above, for example, in which the first conformation is that of a compressed sheet or tube (e.g., a sheet or tube that has been folded, crumpled, flattened and/or rolled). Further related aspects of the invention provide a closure device as described above, for example, in which the second conformation is substantially tubular. Still further related aspects of the invention provide a closure device as described above, for example, in which the body is biodegradable and/or blood-impermeable.

Yet still further related aspects of the invention provide a closure device as described above, for example, in which the body includes shape memory alloys (e.g., nickel titanium, a/k/a Nitinol), shape memory polymers and or other shape memory materials. Still further related aspects of the invention provide a closure device as described above, for example, in which the body includes an expanding membrane which can, in turn, include a shape memory polymer.

The invention provides, in other aspects, a vascular closure device as described above, for example, that includes one or more tethers to maintain the body in the first conformation. Such a tether can, according to related aspects of the invention, be used to release (or otherwise transition) the body into the second conformation. It can also be used, in these and related aspects of the invention, to position and/or maintain position of the body in the vicinity of the vessel wall opening, e.g., while the body is being so transitioned.

Thus, for example, a vascular closure device, e.g., as described above in which the body comprises a tubular member that is compressed (e.g., folded, crumpled, flattened, and/or rolled) in its first conformation, can include a tether that is attached to the body, e.g., at a point substantially half way between its proximal and distal ends. The tether maintains the body in the compressed conformation prior to and during insertion of the body through the opening in a wall of the vessel. Once the body is in place, the tether is removed and the body opens to its second conformation.

Further aspects of the invention provide methods for closing an opening in a vessel. The method can include passing a body that is in a first conformation through the opening in the vessel and, then, transitioning the body from the first conformation to a second conformation.

Related aspects of the invention provide methods as described above in which the step of passing the body through the vessel opening can include inserting a distal end of an introducer sheath through the opening, pushing the closure device out of the distal end of the introducer sheath into the vessel, and withdrawing the introducer sheath from the vessel.

Such methods can further include, according to related aspects of the invention, positioning the body within the vessel so that it (the body) is disposed centrally across the opening.

Further related aspects of the invention provide such methods in which the step of transitioning the body from the first conformation to the second conformation is effected in response to exposing the device to blood temperature and/or hydrating the device. According to further related aspects of the invention, the transitioning step can include releasing a tether and/or allowing a shape memory portion of the device to return to a resting configuration.

These and other aspects of the invention are evident in the drawings and in the text below.

### BRIEF DESCRIPTION OF THE DRAWINGS

The invention will be more fully understood from the following detailed description taken in conjunction with the accompanying drawings, in which:

FIG. 1A is a perspective view of a vascular closure device according to the invention;

FIG. 1B is a perspective view of the vascular closure device of FIG. 1A in a collapsed conformation;

FIG. 1C is a perspective view of the vascular closure device of FIGS. 1A and 1B in a collapsed and rolled conformation;

FIG. 1D is a perspective view of the vascular closure device of FIGS. 1A-1C that is maintained in a collapsed and rolled conformation with a tether;

FIGS. 2-4 illustrate a method of closing an opening in an artery (shown in phantom) according to the invention;

FIG. 5 is a side view of a vascular closure device frame (or skeleton) according to the invention;

FIG. 6 is a side view of the skeleton of FIG. 5 with a membrane defining a continuous outer surface therearound; and

FIG. 7 is a side view of the skeleton of FIG. 5 with a membrane covering only a portion thereof.

### DETAILED DESCRIPTION OF THE ILLUSTRATED EMBODIMENTS

Disclosed herein are devices and related methods for closing of an arteriotomy or arterial wound from the inside. These are discussed in connection with closing catheterization openings in an artery, though, they can be used to close openings of all types in almost any blood vessel or other tubular lumens or ducts in a human or animal body following catheterization, or other medical procedures, disease, trauma or other causes.

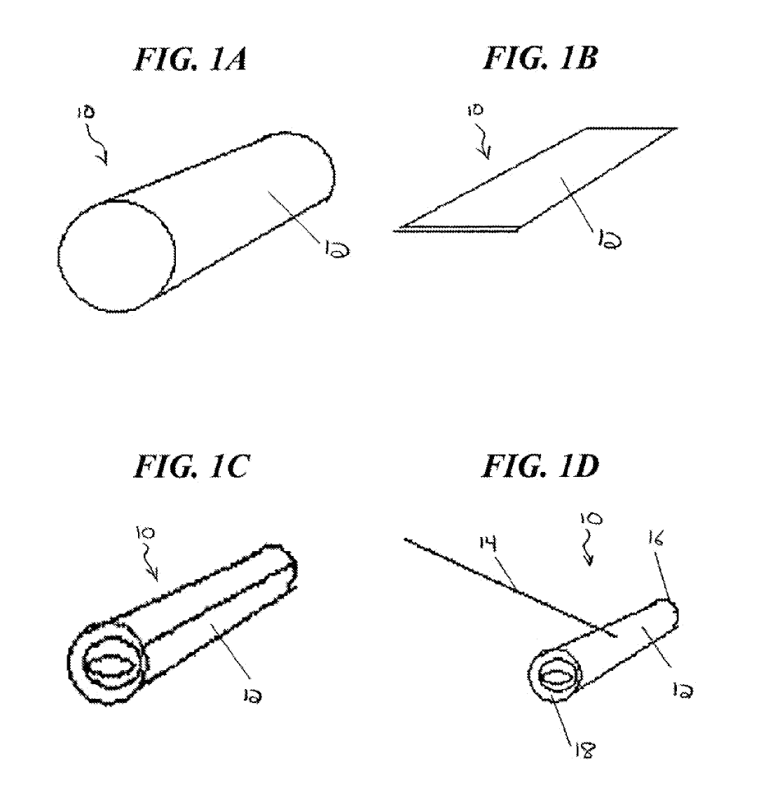

FIGS. 1A-1D illustrate one exemplary embodiment of a vascular closure device 10 according to the invention. As shown, the device 10 generally includes a body 12 and, optionally, at least one tether 14. The body 12 can be formed from a variety of materials, including for example shape memory polymers, shape memory alloys, and/or a combination thereof. The body 12 can be a uniform and homogenous structure or, as explained in more detail below, can be a composite structure that includes a skeleton and a membrane.

The body can have a deployed conformation, as shown in FIG. 1A, in which it is substantially tubular. The body can also have a compressed conformation (that is, a conformation of reduced dimension, e.g., a folded, crumpled, rolled, and/or flattened conformation) such as a collapsed conformation (shown in FIG. 1B) or a collapsed and rolled conformation (shown in FIG. 1C).

The body 12 can be sized and shaped such that it can cover vascular openings of a variety of sizes when in the deployed conformation, including for example an opening having a diameter between 1 mm and 5 mm.

As shown in FIG. 1D, a tether 14 can be attached to the body 12 approximately half way between the proximal end 16 and the distal end 18 of the body 12. The tether 14, which can be a string, wire, suture, or other elongated element can be coupled to the body 12 in a variety of ways. In one embodiment, the tether 14 is looped around the body 12 and tied using a knot. The tether 14 can also be affixed to the body 12 using an adhesive, a friction fit, a snap fit, and/or a variety of other coupling mechanisms or techniques known in the art. The tether 14 can be configured to selectively maintain the body 12 in a particular conformation, e.g., a collapsed and rolled conformation.

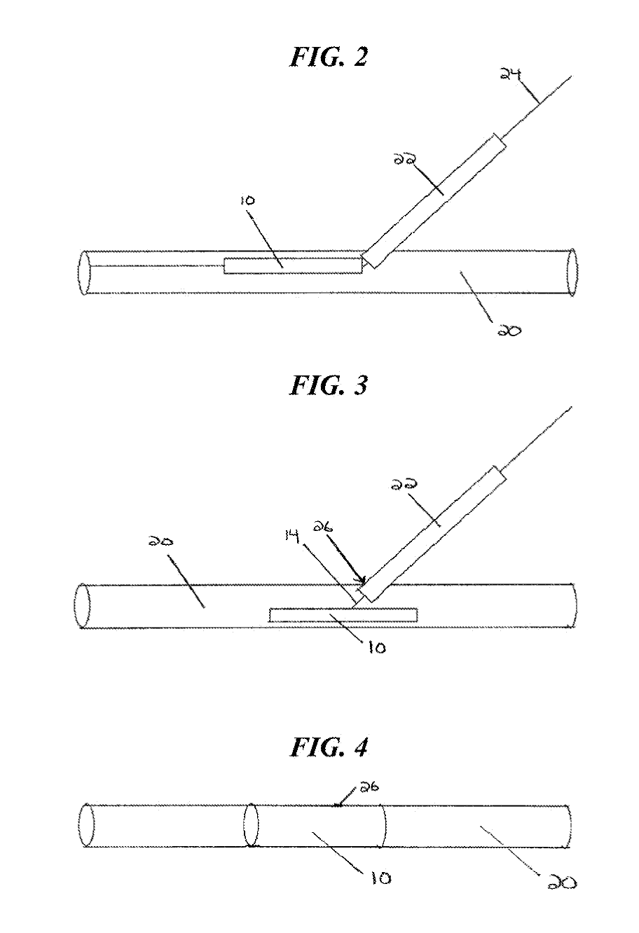

In use, the devices disclosed herein can be configured for delivery into a vessel and, thereafter, for closing an opening the vessel. For example, in one embodiment, a device 10 having a tubular body 12 can be compressed (e.g., here, pressed flat and then rolled into a compressed narrow tubular conformation), as shown for example in FIGS. 1C-1D. The device 10 can then be passed or delivered into a blood vessel 20 through an introducer sheath 22 over a guidewire 24 (or without the guidewire), as shown in FIG. 2. The introducer sheath 22 can be retracted from the blood vessel 20 and the device 10 can also be retracted simultaneously so that it can be positioned centrally over an opening 26 in the vessel 20. The device 10 can be like a stent-graft and can be delivered into the artery in a collapsed form.

The device 10 can be tethered to the introducer sheath 22 via a tether 14, which in one embodiment can be a suture. The device 10 can also be tethered to the external world using a long string or tether, that can be attached to the device in its middle, i.e., not at either end.

After delivering or ejecting the device 10 into the vessel 20 in its collapsed form, the sheath 22 and tether 14 can be retracted which can back the device 10 down into the vessel 20 and position it centrally over the opening 26. In other words, as the sheath 22 is withdrawn or after the sheath 22 is withdrawn, the device 10 can be retracted within the vessel 20 towards the opening 26 such that the device 10 is centrally disposed across the opening 26. Retraction of the device 10 within the vessel 20 can be accomplished by applying tension to a tether or other component coupled thereto. In embodiments in which a tether 14 is coupled to a central portion of the device 10, pulling the tether 14 proximally with respect to the vessel 20 as the body 12 transitions to conform to the vessel wall can ensure that the central portion of the body 12 is aligned with the opening 26 such that the body 12 is positioned centrally over the opening 26 (i.e., the body 12 overlaps the opening 26 by approximately the same amount on all sides).

At that time, the device 10 and the introducer sheath 22 can be positioned with respect to each other in a “T” configuration, as shown for example in FIG. 3. While applying moderate tension to the tether 14, the device 10 can expand, unfurl, and/or unroll in response to a trigger. The trigger can be exposure to body temperature, exposure to moisture, and/or manual release of the tether 14. Other exemplary triggers can include application of electric current and/or application of an electromagnetic field.

In embodiments that include a tether 14, the tether 14 can be a long suture doubled over on itself and incorporated into a restraining mechanism on the closure device 10 which, when one string is released, can be pulled through the device 10 and out of the body like a rip cord, releasing the device 10 so it can expand.

Once the device 10 is fully expanded, it can seal over the access hole or opening 26 from the inside, as shown in FIG. 4, thereby preventing any hemorrhage. In other words, once the trigger is activated (e.g., exposure to blood temperature, hydration in blood for example in the case of shape memory polymers, and/or with release of the tether or “rip cord” for example in the case of shape-memory Nitinol), the tubular construct can expand and seal off the hole from the inside.

The device 10 can optionally be biodegradable, and can thus be designed to degrade over time, for example two to four weeks after deployment within the vessel after the arteriotomy has had sufficient time to heal. The device 10 can be biodegradable over a sufficient time period to allow the artery to heal, but not prolonged enough that the artery develops intimal hyperplasia or narrowing due to proliferative healing.

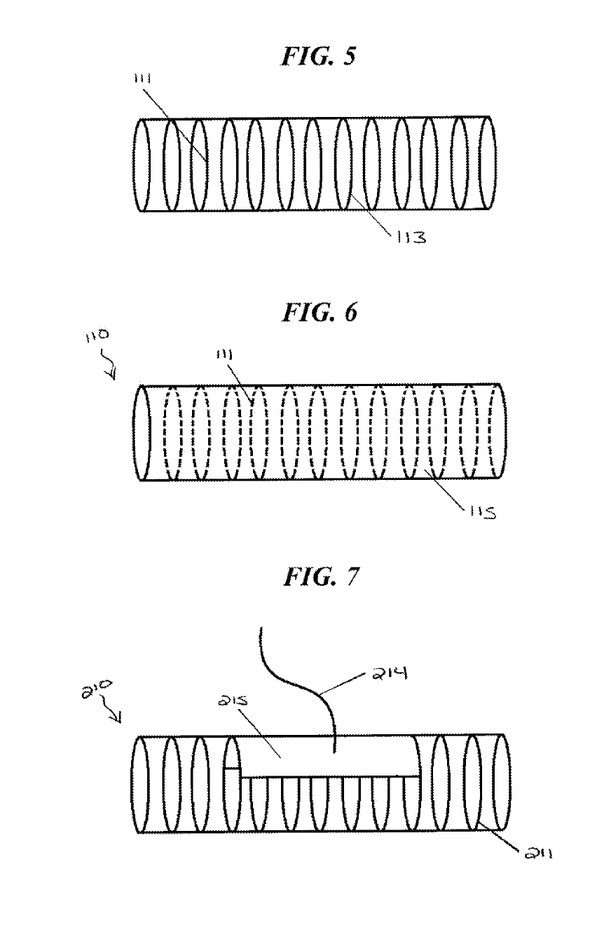

FIGS. 5-6 illustrate another exemplary embodiment of a vascular closure device 110. As shown in FIG. 5, the device can include a skeleton 111 having a plurality of ribs 113 that define a hollow, cylindrical structure. In one embodiment, the skeleton 111 can be formed from a shape memory material such that the device 110 is configured to resiliently return to a pre-determined conformation after being deformed. The skeleton 111 can be formed from a variety of materials that exhibit shape memory properties, including shape memory polymers and shape memory alloys such as Nitinol. The device 110 can also include a skin or membrane 115 disposed over the skeleton 111, as shown for example in FIG. 6. The skin 115 can be formed from a variety of materials, including without limitation materials that are blood-impermeable, substantially blood-impermeable, and/or materials having limited blood permeability.

In another embodiment, as shown in FIG. 7, a vascular closure device 210 is provided in which the skin or membrane 215 covers only a portion of the skeleton 211. In such embodiments, the patch of skin or membrane material 215 can be centrally disposed on the exterior of the skeleton 211. A tether 214 can optionally be attached to the patch 215, for example in the center of the patch, to facilitate placement of the device 210 within a blood vessel. For example, in the illustrate embodiment, pulling the tether 214 through an opening in a blood vessel while the device 210 is disposed within the blood vessel can be effective to pull that patch 215 against the inner wall of the vessel such that it is centrally disposed across the opening to be closed. Although the patch 215 is rectangular in the illustrated embodiment, it can also be round, circular, or virtually any other shape. In one embodiment, the patch 215 can optionally be porous to facilitate in-growth of vascular tissue. In one embodiment, the device (or the portion thereof across which the membrane or skin is disposed) can be sized to cover vascular openings of a variety of sizes, including for example an opening having a diameter between 1 mm and 5 mm.

Described above and shown in the drawings are devices and methods meeting the objects set forth above, among others. Those skilled in the art will understand that those devices and methods are merely examples of the invention and that other embodiments incorporating changes to those shown here fall within the scope of the invention. Thus, for example, a vascular closure kit according to the invention can include multiple vascular closure devices of the types discussed herein that are of varying sizes and conformations, and that respond to varying triggers, etc. as needed in a particular application.

In view of the foregoing, what is claimed is:

1. A vascular closure device, comprising:
a body having a first conformation adaptable to pass through an opening in a vessel and a second conformation adaptable to conform to an inner wall of the vessel in a vicinity of the opening.

2. The device of claim 1, wherein the body is configured to transition from the first conformation to the second conformation in response to a trigger.

3. The device of claim 1, wherein the trigger includes any of exposure to body temperature, exposure to moisture, and release of a tether.

4. The device of claim 1, wherein the body comprises a shape memory polymer.

5. The device of claim 1, wherein the body comprises Nitinol.

6. The device of claim 1, wherein the body comprises an expanding membrane.

7. The device of claim 1, wherein the expanding membrane comprises a shape memory polymer.

8. The device of claim 1, wherein the body is biodegradable.

9. The device of claim 1, wherein the body is blood-impermeable.

10. The device of claim 1, wherein the second conformation is substantially tubular.

11. The device of claim 10, wherein the first conformation is that of a compressed tube.

12. The device of claim 11, wherein the first conformation is that of a flattened and rolled tube.

13. The device of claim 1, wherein the second conformation is that of a sheet.

14. The device of claim 13, wherein the first conformation is that of a rolled sheet.

15. The device of claim 1, further comprising at least one tether configured to selectively maintain the body in the first conformation.

16. The device of claim 15, wherein the tether is further configured to position the device over the opening in the vessel while the device is disposed in the vessel and while the device is in the second conformation.

17. The device of claim 15, wherein the tubular body includes proximal and distal ends and the tether is attached to the body at a point substantially half way between the proximal and distal ends.

18. The device of claim 1, wherein the body comprises a frame formed of a shape memory material and a membrane of limited blood permeability disposed around at least a portion of the frame.

19. The device of claim 18, wherein the membrane is disposed around the entire frame such that the membrane is in the form of a tube.

20. The device of claim 18, wherein the membrane is only disposed around an area of the frame oriented to the opening in the vessel.

21. The device of claim 18, wherein the shape memory material comprises Nitinol.

22. The device of claim 21, wherein the membrane comprises a fabric.

23. The device of claim 18, wherein the shape memory material comprises a shape memory polymer.

24. The device of claim 23, wherein the membrane comprises a shape memory polymer.

25. A method for closing an opening in a vessel, comprising:
providing a closure device having a first conformation adaptable to pass through an opening in a vessel and a second conformation adaptable to conform to an inner wall of the vessel in a vicinity of the opening;
inserting the closure device while in the first conformation through an opening in a vessel;
transitioning the closure device from the first conformation to the second conformation while the closure device is disposed within the vessel and adjacent to the opening.

26. The method of claim 25, wherein inserting the closure device comprises:
inserting a distal end of an introducer sheath through the opening in the vessel;
pushing the closure device out of the distal end of the introducer sheath into the vessel; and
withdrawing the introducer sheath from the vessel.

27. The method of claim 26, wherein inserting the closure device further comprises retracting the closure device within the vessel such that the closure device is disposed centrally across the opening.

28. The method of claim 25, further comprising compressing the closure device to transition the device to the first conformation before inserting it through the opening.

29. The method of claim 28, wherein compressing the closure device comprises collapsing the closure device and then rolling the collapsed closure device.

30. The method of claim 25, wherein transitioning the closure device from the first conformation to the second conformation comprises exposing the device to blood temperature.

31. The method of claim 25, wherein transitioning the closure device from the first conformation to the second conformation comprises hydrating the device with blood.

32. The method of claim 25, wherein transitioning the closure device from the first conformation to the second conformation comprises actuating a tether.

33. The method of claim 25, wherein transitioning the closure device from the first conformation to the second conformation comprises allowing a shape memory portion of the device to return to a resting configuration.

34. The method of claim 25, wherein actuating the tether comprises pulling the tether proximally away from the closure device.

35. The method of claim 25, wherein the closure device is at least one of blood-impermeable, biodegradable, and tubular.
